(19) United States
(12) Patent Application Publication
(10) Pub. No.: US 2021/0189507 A1
(43) Pub. Date: Jun. 24, 2021

(54) PRIMER, KIT AND METHOD FOR DETECTING TAIHE BLACK CHICKEN EGG

(71) Applicant: Nanchang Normal University, Nanchang (CN)

(72) Inventors: Jiguo Xu, Nanchang (CN); Yousheng Rao, Nanchang (CN); Min Zhou, Nanchang (CN); Jing'e Ma, Nanchang (CN); Xuenong Zhu, Nanchang (CN); Yuwen Tan, Nanchang (CN); Jishang Gong, Nanchang (CN); Yanbei Yang, Nanchang (CN); Yuanfei Li, Nanchang (CN); Qiao Xu, Nanchang (CN); Xinwei Xiong, Nanchang (CN); Zhangfeng Wang, Nanchang (CN); Zhiwu Chen, Nanchang (CN); Tingchong Chen, Nanchang (CN); Jianjun Peng, Nanchang (CN)

(21) Appl. No.: 17/122,055

(22) Filed: Dec. 15, 2020

(30) Foreign Application Priority Data
Dec. 19, 2019 (CN) 2019113189635

(51) Int. Cl. C12Q 1/6888; C12Q 1/6806

(52) U.S. Cl. CPC C12Q 1/6888; C12Q 2531/113; C12Q 2563/179; C12Q 1/6806

(57) ABSTRACT

This invention discloses a primer, a kit and a method for testing Taihe black chicken egg. The described primer includes: as shown in SEQ ID No. 1, SEQ ID No. 2, SEQ ID No. 3 and SEQ ID No. 4. The described test method comprises the following steps: extract the genomic DNA of the egg to be tested, propagate the genomic DNA by two-step PCR, and perform genotyping according to the base at 68 bp of the gene sequence of the second-step PCR product. If the base at that site is G, the egg is a Taihe black chicken egg; if the base at that site is C, the egg is a non-Taihe black chicken egg. The variable site targeted by this invention is unique to Taihe black chicken egg.

### FIELD OF THE INVENTION

This invention relates to the technical field of molecular biodetection, in particular to a primer, a kit and a method for testing Taihe black chicken egg.

### REFERENCE TO A SEQUENCE LISTING

This application refers to a “Sequence Listing” listed below which is provided as an electronic document entitled “Sequence Listing.txt” (1.31 kb, created on Jan. 21, 2021) which is incorporated herein in its entirety.

### BACKGROUND OF THE INVENTION

Taihe black chicken farming is the most distinctive advantageous industry of the economic development of Taihe County, and has become a name card of Taihe County's agricultural sector. Taihe black chicken has been well-known since ancient times. With the economic and social development and the improvement of people's living standards, people's demand for high-quality, safe and healthy food is increasing. The striking quality and price of Taihe black chicken egg leads to the gradual increase of fake black chicken eggs, but most consumers do not have the ability to identify Taihe black chicken egg. It provides many illegal businessmen with the opportunity of illegal operation, which is not conducive to the standardization of the overall market.

At present, there is no effective method to identify Taihe black chicken egg. The two most common methods are: [symbol] the business firm labels the egg; [symbol] establish a product traceability system to track and record products for the whole course through RFID, QR code or barcode technology. However, all the above means are based on the integrity of the merchants, and unscrupulous businessmen can still pass off non-black chicken eggs as Taihe black chicken eggs by technical means. Our aim is to protect the healthy development of Taihe black chicken industry, stabilize the quality of Taihe black chicken eggs, standardize the market of black chicken eggs, and prevent non-black chicken eggs from passing off as high quality and expensive black chicken eggs. Gene detection technology can be used to directly test the base of DNA molecules and has a high accuracy. Therefore, it is very urgent to establish a method for identifying Taihe black chicken egg by using gene detection technology. The establishment of the molecular detection method for identifying Taihe black chicken egg can not only crack down on counterfeiters and prevent unscrupulous vendors from messing up the black chicken egg market, but also enhance the image of Taihe black chicken and further enhance the brand value of Taihe black chicken.

### SUMMARY OF THE INVENTION

The purpose of this invention is to provide a primer, a kit and a method for testing Taihe black chicken egg, by which sufficient PCR product for sequencing and typing can be obtained through a two-step propagation of the genomic DNA of the egg by using the primer, and true and false Taihe black chicken egg can be further judged accurately through the identification of the variable site of the PCR product.

To achieve the above purpose, this invention provides the following scheme:

This invention provides a primer for testing Taihe black chicken egg, including: as shown in SEQ ID No. 1, SEQ ID NO. 2, SEQ ID No. 3 and SEQ ID No. 4.

This invention also provides a method for testing Taihe black chicken egg, which comprises the following steps: extract the genomic DNA of the egg to be tested, propagate the described genomic DNA by two-step PCR, and perform genotyping according to the base at 68 bp of the gene sequence of the obtained second-step PCR product. If the base at that site is G, the egg is a Taihe black chicken egg; if the base at that site is C, the egg is a non-Taihe black chicken egg. Preferably, in the first-step PCR propagation, the primer is SEQ ID NO. 1 and SEQ ID NO. 2 according to claim 1;

30 μL, propagation system: the template DNA is 2 μL, 2×PCR mix is 15 μL, mixed primer is 0.6 μL, and the rest is water;

Propagation procedure: 95° C. for 3 min; 94° C. for 15 s, 55° C. for 30 s, 72° C. for 20 s, 20 cycles; 72° C. for 3 min.

Preferably, in the second-step PCR propagation, the primer is SEQ ID NO. 3 and SEQ ID NO. 4 according to claim 1;

20 μL, propagation system: the first PCR product is 1 μL, 2×PCR mix is 10 μL, mixed primer is 0.4 μL, and the rest is water;

Propagation procedure: 96° C. for 3 min; 96° C. for 30 s, 52° C. for 30 s, 72° C. for 30 s, 35 cycles.

This invention also provides the application of the described method for detecting Taihe black chicken egg in identifying Taihe black chicken egg.

This invention also provides a kit containing the described primer for detecting Taihe black chicken egg.

This invention also provides a described kit identifying true and false Taihe black chicken egg.

This invention discloses the following technical effects:

Based on the polymorphism of 68 bp site of the sequence of the second-step PCR product, this invention determines the allele of that site by gene sequencing method. If the site is G, the egg is determined as Taihe black chicken egg; if site is C, the egg is determined as non-Taihe black chicken egg. This method can effectively prevent illegal vendors from illegal operation of passing off non-Taihe black chicken eggs as Taihe black chicken eggs to sell to and cheat consumers.

The two-step PCR propagation is used in the testing method provided by this invention. As an egg, especially the ovule of an unfertilized egg is only a cell, tissue extraction will lead to the complete loss of the only DNA, therefore, it is difficult to extract enough DNA for PCR. In this invention, the ovule is broken by physical method and releases genomic DNA instead of being extracted, and then a sufficient amount of PCR product for sequencing is obtained by a two-step PCR propagation. After that, it is typed by sequencing to accurately obtain the genotype of the variable site. With the testing method disclosed by this invention, the SNP site to be tested is the unique variable site of Taihe black chicken. This method can identify whether the tested individual egg is a Taihe black chicken egg with an accuracy of 100%, and it can be ruled out that unscrupulous vendors pass off non-Taihe black eggs as Taihe black eggs. This method is simple, easy to operate and has high detection accuracy.

### BRIEF DESCRIPTION OF THE DRAWINGS

In order to more clearly explain the embodiments of this invention or the technical scheme in the prior art, the drawings needed in the embodiments will be briefly introduced as below. Obviously, the drawings described below are only some embodiments of this invention. For those of ordinary skill in the art, other drawings can be obtained from these drawings without making creative effort.

FIG. 1 shows the result of variable site typing of Taihe black chicken egg.

### DETAILED DESCRIPTION OF THE INVENTION

Various exemplary embodiments of this invention will be described in detail, which should not be considered as a limitation of this invention, but as a more detailed description of certain aspects, characteristics and embodiments of this invention.

It should be understood that the terms used in this invention are only for describing particular embodiments and are not intended to limit this invention. In addition, for the numerical range in this invention, it should be understood that each intermediate value between the upper and lower limits of the range is also specifically disclosed. Each smaller range between any stated value or the intermediate value within the stated range and any other stated value or the intermediate value within the described range is also included in this invention. The upper and lower limits of these smaller ranges can be included or excluded from the range independently.

Unless otherwise indicated, all technical and scientific terms used herein have the same meaning commonly understood by those of ordinary skill in the art of this invention. Although this invention only describes preferred methods and materials, any methods and materials similar to or equivalent to those described herein may also be used in the embodiments or tests of this invention. All documents in these specifications are incorporated by reference to disclose and describe the methods and/or materials related to the described documents. In case of conflict with any incorporated document, the content of these specifications shall prevail.

Without deviating from the scope or spirit of this invention, various improvements and changes can be made to the specific embodiments of the specifications of this invention, which is obvious to those skilled in the art. Other embodiments obtained from the specifications of this invention are obvious to the technicians. The specifications and embodiments of this application are exemplary only.

The words “contain”, “include”, “possess”, “have” and so on used in this paper are all open terms, that is, they mean to include but not limited to.

Unless otherwise specified, the “parts” mentioned in this invention refers to “parts by mass”.

### Embodiment 1

A method for testing Taihe black chicken egg, which comprises the following steps:

1. Extraction of sample DNA

(1) Break the shell of the egg to be tested, separate the egg white from the yolk, and place the yolk in a plate with PBS.

(2) Take the ovule or blastoderm (white, with a diameter of about 2 mm) on the vitelline membrane, cut the vitelline membrane around the ovule or blastoderm with a scalpel, place the ovule or blastoderm in a 2 mL centrifuge tube, and then place the tissue in a refrigerator at −20° C. or liquid nitrogen.

(3) Tissue crushing: add appropriate amount of liquid nitrogen into the 2 mL centrifuge tube with ovule or blastoderm tissue, and crush it with tissue crusher.

(4) Add 0.2 mL double distilled water to the crushed tissue and perform vortex oscillation for 30 s.

(5) Centrifuge it at 13000 r/min for 10 min, take the supernatant and put it into another centrifuge tube.

2. PCR propagation

2.1 The first-step PCR propagation

Dissolve the primes SF1: cccccactcagagcgctctg (SEQ ID NO: 1) and SR1: ggcggaccggcgcggcctta (SEQ ID NO: 2) to 10 pmoL with 1×TE respectively, mix the primers together, and perform vortex mixing evenly.

Prepare 30 μL PCR propagation system (as shown in Table 1)

TABLE 1

| Kit | The dosage |
|---|---|
| the template DNA | 2 μL |
| 2*PCR mix | 15 μL |
| mixed primer | 0.6 μL |

H₂0 supplement to 30 μL

Divide the prepared product in PCR header into a 96-well PCR plate, centrifuge it, and then add 2 μL the DNA sample into each well, centrifuge it, apply PCR instrument, and obtain the propagated product. The gene sequence of the PCR product was as follows (The target site is shown in [symbol] [symbol] )

(SEQ ID NO: 5)
cccccactcagagcgctctgcgactctcaacgcgggaacgccgcga
gaggccgtcaggcgcggaagacgagcgaagcgggaagggagagccg
cgctgcctcgcttta [symbol] c/g [symbol] ggcccgtgtgcgacgcgcaagatg
gctgcccccagggcgcaataaggccgcgccggtccgcc.

The conditions for the above PCR propagation were as follows: 95° C. for 3 min; 94° C. for 15 s, 55° C. for 30 s, 72° C. for 20 s, 20 cycles; 72° C. for 3 min.

2.2 The second-step PCR propagation

Take the PCR product obtained from the first propagation as the template and make use of the primer

SF2:
(SEQ ID NO: 3)
ccgcgagaggccgtcaggcg,
SR2:
(SEQ ID NO: 4)
ttgcgccctgggggcagcca,

perform the second PCR propagation by using the 20 μL propagation system to obtain propagation product. The sequence of the PCR product was as follows (The target site is shown in [symbol] [symbol] )

(SEQ ID NO: 6)
ccgcgagaggccgtcaggcgcggaagacgagcgaagcgggaaggga
gagccgcgctgcctcgcttta [symbol] c/g [symbol] ggcccgtgtgcgacgcgc
aagatggctgcccccagggcgcaa.

Prepare the 20 μL propagation system (as shown in Table 2).

TABLE 2

| Kit | The dosage |
|---|---|
| the template DNA | 2 μL |
| 2*PCR mix | 10 μL |
| mixed primer | 0.4 μL |

H₂0 supplement to 20 μL

Propagation conditions: 96° C. for 3 min; 96° C. for 30 s, 52° C. for 30 s, 72° C. for 30 s, 35 cycles.

2.3 Send the PCR product obtained from the second propagation to the biotech company for sequencing. Read the experimental results by the SeqMan module of DNAStar software and check the genotype of the target site.

2.4 Result analysis

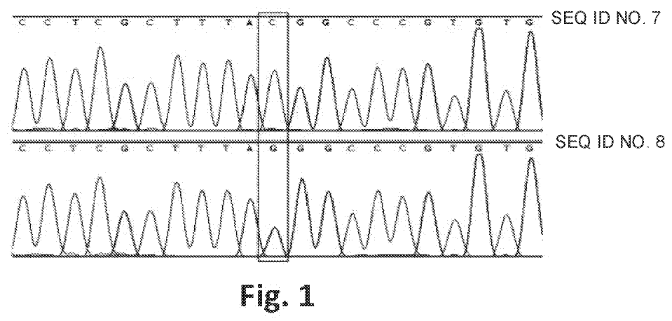

If the variable site was GG genotype, the egg was a Taihe black chicken egg, otherwise it was a non-Taihe black chicken egg (as shown in FIG. 1).

### Embodiment 2

Taihe black chicken eggs and non-Taihe black chicken eggs (white ear yellow chicken eggs, Ningdu yellow chicken eggs, Kangle yellow chicken eggs and Anyi dark gray chicken eggs) were identified by the testing method in Embodiment 1 with 10 tested eggs for each variety.

As shown in Table 3, the results showed that: Only the base of the variable site of Taihe black chicken eggs was G, and that of all other eggs was C. The result was consistent with the actual situation.

TABLE 3
Typing results of different eggs

| Egg variety | Genotype |
|---|---|
| Taihe black chicken eggs (10) | G |
| White ear yellow chicken eggs (10) | C |
| Ningdu yellow chicken eggs (10) | C |
| Kangle yellow chicken eggs (10) | C |
| Anyi dark gray chicken eggs (10) | C |

### Embodiment 3

Pure Taihe black chicken eggs (Group A) and eggs mixed with non-Taihe black chicken eggs (Group B) were identified by the testing method in Embodiment 1. Thirty Taihe black chicken eggs were selected and mixed with 30 non-Taihe black chicken eggs.

Type the eggs by the testing method in Embodiment 1 and count the site gene frequency of each egg group. If the egg was pure Taihe black chicken egg, the frequency of G allele review should be 100%, otherwise it was a certain value of <100%.

As shown in Table 4, the results showed that the G allele frequency in Group A was 100%, that in Group B was 57%, and that in Group C was 43%. According to the statistical results, Group A was determined as Taihe black chicken eggs, and Group B was mixed black chicken eggs.

TABLE 4
Determination results of two groups of eggs

| | Allele frequency | | Statistical |
|---|---|---|---|
| Group | G | C | result |
| A | 100% (30) | 0% (0) | Taihe black chicken eggs |
| B | 57% (17) | 43% (13) | Mixed black chicken eggs |

From the testing results of Embodiment 2-3, it can be seen that the testing method provided by this invention can not only accurately identify Taihe black chicken eggs and non-Taihe black chicken eggs, but also identify whether there are non-Taihe black chicken eggs mixed in the so-called “Taihe black chicken eggs” on the market, which can truly regulate the market at the genetic level and prevent illegal vendors from passing off non-Taihe black chicken eggs or non-pure Taihe black chicken eggs as Taihe black chicken eggs, thus damaging the interests of consumers.

The embodiments described above describe the preferred modes of this invention only, not limit the scope of this invention. Without deviating from the design spirit of this invention, all modifications and improvements made by ordinary technicians in the art to the technical scheme of this invention shall fall into the protection scope determined by the claims of this invention.

1. A primer for testing Taihe black chicken egg, characterized in that it comprises: SF1: cccccactcagagcgctctg, SR1: ggcggaccggcgcggcctta, SF2: ccgcgagaggccgtcaggcg, and SR2: ttgcgccctgggggcagcca.

2. A method for detecting Taihe black chicken egg, characterized in that it comprises the following steps: extract the genomic DNA of the egg to be tested, perform the first-step PCR propagation with primer SF1: cccccactcagagcgctctg, SR1: ggcggaccggcgcggcctta, perform the second PCR propagation with the PCR product obtained from the first propagation as the template by using primer SF2: ccgcgagaggccgtcaggcg, SR2: ttgcgccctgggggcagcca, and perform genotyping according to the base at 68 bp of the gene sequence of the obtained second-step PCR product. If the base at that site is G, the egg is a Taihe black chicken egg; if the base at that site is C, the egg is a non-Taihe black chicken egg.

3. The method for detecting Taihe black chicken egg according to claim 2 is characterized in that in the first-step PCR propagation, the primer is SF1: tcagagcgctctg, SR1: ggcggaccggcgcggcctta according to claim 1;
30 μL, propagation system: the template DNA is 2 μL, 2×PCR mix is 15 μL, mixed primer is 0.6 μL, and the rest is water;
Propagation procedure: 95° C. for 3 min; 94° C. for 15 s, 55° C. for 30 s, 72° C. for 20 s, 20 cycles; 72° C. for 3 min.

4. The method for detecting Taihe black chicken egg according to claim 2 is characterized in that in the second-step PCR propagation, the primer is SF2: aggccgtcaggcg, SR2: ttgcgccctgggggcagcca according to claim 1;
20 μL, propagation system: the first PCR product is 1 μL, 2×PCR mix is 10 μL, mixed primer is 0.4 μL, and the rest is water;
Propagation procedure: 96° C. for 3 min; 96° C. for 30 s, 52° C. for 30 s, 72° C. for 30 s, 35 cycles.

5. The application of the method for detecting Taihe black chicken egg according to claim 2 in identifying Taihe black chicken egg.

6. A kit containing the primer for detecting Taihe black chicken egg according to claim 1.

7. The application of the kit according to claim 6 in identifying true and false Taihe black chicken egg.


SEQUENCE LISTING (8 sequences)

SEQ ID NO: 1 - DNA, 20 bp, Artificial Sequence
  Synthetic forward primer sequence
cccccactcagagcgctctg

SEQ ID NO: 2 - DNA, 20 bp, Artificial Sequence
  Synthetic reverse primer sequence
ggcggaccggcgcggcctta

SEQ ID NO: 3 - DNA, 20 bp, Artificial Sequence
  Synthetic forward primer sequence
ccgcgagaggccgtcaggcg

SEQ ID NO: 4 - DNA, 20 bp, Artificial Sequence
  Synthetic reverse primer sequence
ttgcgccctgggggcagcca

SEQ ID NO: 5 - DNA, 170 bp, Artificial Sequence
  Gene sequence of PCR product
cccccactcagagcgctctgcgactctcaacgcgggaacgccgcgagaggccgtcaggcgcggaagacgagcgaagcgggaagggagagccgcgctgcctcgctttasggcccgtgtgcgacgcgcaagatggctgcccccagggcgcaataaggccgcgccggtccgcc

SEQ ID NO: 6 - DNA, 110 bp, Artificial Sequence
  Gene sequence of PCR product
ccgcgagaggccgtcaggcgcggaagacgagcgaagcgggaagggagagccgcgctgcctcgctttasggcccgtgtgcgacgcgcaagatggctgcccccagggcgcaa

SEQ ID NO: 7 - DNA, 21 bp, Gallus gallus
cctcgctttacggcccgtgtg

SEQ ID NO: 8 - DNA, 21 bp, Gallus gallus
cctcgctttagggcccgtgtg